(19) United States
(12) Patent Application Publication
(10) Pub. No.: US 2025/0275691 A1
(43) Pub. Date: Sep. 4, 2025

(54) FLEXIBLE MAGNETOELECTRIC PIEZOELECTRIC SWITCH APPARATUS, WEARABLE PATCH, AND METHOD FOR PREPARING AND APPLICATION THEREOF

(71) Applicants: JS Nanotechnologies LLC, San Jose, CA (US); Hubei University of Technology, Wuhan (CN)

(72) Inventors: Jeongmin Hong, San Jose, CA (US); Zhiyi Sun, Wuhan (CN)

(21) Appl. No.: 19/070,425

(22) Filed: Mar. 4, 2025

(30) Foreign Application Priority Data
Mar. 4, 2024 (US) 63561015

(51) Int. Cl. A61B 5/11; A61M 35/00; H01F 1/00; H10N 30/30

(52) U.S. Cl. CPC A61B 5/1126; A61M 35/10; H01F 1/0054; A61B 2562/0223; A61B 2562/0285; A61M 2205/0294; H10N 30/302

(57) ABSTRACT

A flexible magnetoelectric piezoelectric switch apparatus includes a thin film flexible substrate. The thin film flexible substrate includes: a flexible substrate, a piezoelectric substrate, and magnetoelectric nano particles. The magnetoelectric nano particles are of a core-shell structure, including a cobalt ferrite magnetic core and a barium titanate piezoelectric shell layer. The magnetoelectric nano particles are dispersed on a surface of the piezoelectric substrate. The apparatus further includes a transducer and a sensor. The flexible magnetoelectric piezoelectric switch apparatus is obtained by coupling the transducer, the thin film flexible substrate, and the sensor sequentially.

### TECHNICAL FIELD

The present disclosure relates to the technical field of sensors, in particular to a flexible magnetoelectric piezoelectric switch apparatus, a wearable patch, and a method for preparing and application thereof.

### BACKGROUND

Multiferroic materials are of interest due to the coupling of different ferrous orders and their magnetoelectric effects. Such materials include substances with characteristics such as (anti) ferromagnetism, ferroelectricity, and ferroelasticity, and have become a research hotspot driven by the demand for a new generation of high-performance electronic devices. Novel nano particles (NPs) based on ferroelectric (FE) and ferromagnetic (FM) characteristics, namely multiferroic nano particles (MFNPs), have been discovered and used for carrier transportation and sensing. Unlike conventional magnetic NPs, composite multiferroic nano particles (MFNPs) of a core/shell structure may achieve low-field on-demand driving due to their magnetoelectric coupling effect, thus having advantages of low loss and high energy efficiency. This makes a nano-controlled release system based on a magnetoelectric nano particle (MENP) promising for sensing and driving applications in a biological environment, especially in a wearable or implantable system.

### SUMMARY

A technical solution of the present disclosure is implemented by the following steps.

In a first aspect, the present disclosure provides a flexible magnetoelectric piezoelectric switch apparatus, including a thin film flexible substrate. The thin film flexible substrate includes: a flexible substrate, a piezoelectric substrate, and MENPs.

The MENPs are of a core-shell structure, including a cobalt ferrite magnetic core and a barium titanate piezoelectric shell layer. The MENPs are dispersed in the piezoelectric substrate.

In a second aspect, the present disclosure relates to a method for preparing the above flexible magnetoelectric piezoelectric switch apparatus, including the following step: obtaining the flexible magnetoelectric piezoelectric switch apparatus by coupling a transducer, the thin film flexible substrate, and a sensor sequentially.

In a third aspect, the present disclosure provides a wearable patch, including the above flexible magnetoelectric piezoelectric switch apparatus.

### BRIEF DESCRIPTION OF DRAWINGS

To describe the technical solutions in the embodiments of the present invention or in the related art more clearly, the following briefly introduces the accompanying drawings for describing the embodiments or the related art. Apparently, the accompanying drawings in the following description show merely some embodiments of the present invention, and a person of ordinary skill in the art may still derive other drawings from the accompanying drawings without creative efforts.

FIG. 1 is a schematic diagram of a PVDF thin film, associated images thereof, and a thin film flexible substrate provided by one or some embodiments of the present application.

FIG. 2 is a simulated electric field and potential diagram generated by a PVDF film provided by one or some embodiments of the present application.

FIG. 3 is a diagram of samples of a PVDF film after polarization provided by one or some embodiments of the present application.

FIG. 4 is an image of deposited magnetic nano particles and a deposition process provided by one or some embodiments of the present application.

FIG. 5 is a diagram of changes in electric field distribution at different distances between magnetic nano particles and a PVDF film provided by one or some embodiments of the present application.

FIG. 6 is an image of magnetic nano particles based on a magneto-optical Kerr effect (MOKE) provided by one or some embodiments of the present application.

FIG. 7 is a diagram of an M-H hysteresis loop of CoFe₂O₄ present in forms of nano particles and a thin film provided by one or some embodiments of the present application.

FIG. 8 are M-H hysteresis loops obtained for magnetic nano particles under different stress conditions provided by one or some embodiments of the present application.

FIG. 9 is a diagram of magnetization intensity change curves and distance dependence curves under different stresses provided by one or some embodiments of the present application.

FIG. 10 is a diagram of polarization phenomena of a PVDF film under different stress conditions provided by one or some embodiments of the present application.

FIG. 11 is a diagram of voltage changes during a polarization process of a PVDF film provided by one or some embodiments of the present application.

FIG. 12 is a diagram of characteristics of a magnetic phase change of a PVDF film provided by one or some embodiments of the present application.

FIG. 13 is a diagram of responses of MENPs to voltage changes provided by one or some embodiments of the present application.

FIG. 14 is an XRD diagram of a P(VDF-TrFE) thin film under different sintering conditions provided by one or some embodiments of the present application.

FIG. 15 is a diagram of a soft piezoelectric device provided by one or some embodiments of the present application.

FIG. 16 is a diagram of physical characteristics of an MENP device provided by one or some embodiments of the present application.

FIG. 17 is a structural diagram and a preparation diagram of an MENP patch provided by one or some embodiments of the present application.

FIG. 18 is a diagram of performance of an MENP patch provided by one or some embodiments of the present application.

FIG. 19 is a schematic diagram of a rotary nanopatch provided by one or some embodiments of the present application.

FIG. 20 is a schematic diagram of a nanopatch assembly provided by one or some embodiments of the present application.

FIG. 21 is a schematic diagram of three nanopatch structures provided by one or some embodiments of the present application.

FIG. 22 are nanopatch products provided by one or some embodiments of the present application.

FIG. 23 is a schematic diagram of a system containing a PVDF film provided by one or some embodiments of the present application.

FIG. 24 is a schematic diagram of a system containing a magnetoelectric effect nano particle sensor patch provided by one or some embodiments of the present application.

FIG. 25 is a schematic flowchart of a method 1200 provided by one or some embodiments of the present application.

FIG. 26 is a schematic diagram of functions of a system of a nano particle sensor patch provided by one or some embodiments of the present application.

FIG. 27 is a diagram of characteristics of a nano particle sensor patch provided by one or some embodiments of the present application.

FIG. 28 is a schematic diagram of operation of a nano particle sensor patch provided by one or some embodiments of the present application.

FIG. 29 is a schematic diagram of a working mechanism of a touch sensor provided by one or some embodiments of the present application.

FIG. 30 is a diagram of structural characteristics of different layers of a nano particle sensing patch provided by one or some embodiments of the present application.

FIG. 31 is a diagram of a sensor actuation mechanism for a nano particle sensing patch provided by one or some embodiments of the present application.

FIG. 32 is a diagram of applications of a nano particle sensing patch provided by one or some embodiments of the present application.

FIG. 33 is a diagram of associated detection of a nano particle sensing patch provided by one or some embodiments of the present application.

FIG. 34 is a nano particle sensing patch for a drug release application provided by one or some embodiments of the present application.

FIG. 35 is a diagram of a drug release process for a nano particle sensing patch provided by one or some embodiments of the present application.

FIG. 36 is a diagram illustrating characteristics for a drug delivery by a nano particle sensing patch provided by one or some embodiments of the present application.

FIG. 37 is a diagram of an inkjet printing process provided by one or some embodiments of the present application.

FIG. 38 is a diagram of inkjet printing details provided by one or some embodiments of the present application.

FIG. 39 is a diagram illustrating deposition characteristics of an MENP droplet provided by one or some embodiments of the present application.

FIG. 40 is a diagram of a moving position of a droplet of a particle solution provided by one or some embodiments of the present application.

FIG. 41 is a diagram of an edge region of a droplet provided by one or some embodiments of the present application.

FIG. 42 is a diagram of uniform droplet deposition on a flexible piezo-element provided by one or some embodiments of the present application.

FIG. 43 is a diagram of uniform droplet deposition on a flexible substrate provided by one or some embodiments of the present application.

FIG. 44 is a diagram of moving positions of small particles and big particles provided by one or some embodiments of the present application.

FIG. 45 is a flowchart of spray-coating magnetic particles provided by one or some embodiments of the present application.

FIG. 46 is a diagram of details of spray-coating magnetic particles provided by one or some embodiments of the present application.

FIG. 47 is a diagram of a process for preparing a nano particle sensing patch provided by one or some embodiments of the present application.

FIG. 48 is a diagram of particle deposition and a magnetic field change provided by one or some embodiments of the present application.

FIG. 49 is an example particle type provided by one or some embodiments of the present application.

FIG. 50 is inkjet printing of a P(VDF-TrFE) film provided by one or some embodiments of the present application.

FIG. 51 is a diagram of sintering data for P(VDF-TrFE) inkjet printing provided by one or some embodiments of the present application.

FIG. 52 is a diagram of a specific scenario of P(VDF-TrFE) inkjet printing provided by one or some embodiments of the present application.

FIG. 53 is a structural diagram of different layers of a nano particle sensing patch provided by one or some embodiments of the present application.

FIG. 54 is images of a nano particle sensing patch under different magnifications provided by one or some embodiments of the present application.

FIG. 55 is a diagram of response characteristics of different types of sensors provided by one or some embodiments of the present application.

FIG. 56 is a diagram of characteristics of different wearable devices provided by one or some embodiments of the present application.

FIG. 57 is a flowchart for preparing a flexible magnetoelectric piezoelectric switch apparatus provided by one or some embodiments of the present application.

### DETAILED DESCRIPTION

The technical solutions in the embodiments of the present invention will be clearly and completely described below with reference to the embodiments of the present invention, and it is obvious that the described embodiments are only a part of the embodiments of the present invention but not all of them. All other embodiments obtained by a person of ordinary skill in the art based on the embodiments of present disclosure without making creative efforts shall fall within the protection scope of present disclosure.

For relevant performance tests below, the following special notes are made:

### Simulation Experiment

COMSOLMultiphysics was used to simulate a strain-induced magnetoelectric (ME) effect based on a thin film flexible substrate, wherein a flexible substrate was a 10 μm×10 μm×1 μm cuboid, and MENPs had a core radius of 0.1 μm and a shell layer radius of 0.2 μm.

In a simulation model, applying a stress to the flexible substrate generated an electric field on its exterior. Under the action of this electric field, ferromagnetic materials of shell layers of the MENPs were strained due to an electrostriction effect, and this strain was further transferred to ferromagnetic materials of an inner core, thus changing its magnetization state through an inverse magnetostriction effect, i.e., an ME effect.

### (2) SEM and TEM Characterizations

A TEM image was acquired using a transmission electron microscope (TEM) with an acceleration voltage of 200 kV. The experiment used a field emission gun equipped with an imaging filter (Gatan, GIF200) and running DigitalMicrograph™ software. A TEM sample was prepared by dropping a small emulsion on a carbon-coated copper (Cu) grid and performing drying under ambient conditions.

### (3) Scanning Probe Microscope (SPM)

A scanning electron microscope (SEM, JEOL9000F) was used for an characterization at an acceleration voltage of 15 kV and a working distance of 5 mm. SPM study was performed in non-contact mode using a Bruker-NanoAFM system. A measurement of a magnetic force microscope (MFM) used a dynamic lifting mode, and modulation is performed by adjusting a lifting distance.

The present application provides a flexible magnetoelectric piezoelectric switch apparatus, including a thin film flexible substrate. The thin film flexible substrate includes: a flexible substrate, a piezoelectric substrate, and MENPs.

The flexible substrate includes a PDMS thin film, an AgNW network, and a polyvinylidene fluoride film.

The piezoelectric substrate is a (P(VDF-TrFE)) thin film.

The MENPs are of a core-shell structure, including a cobalt ferrite magnetic core and a barium titanate piezoelectric shell layer. The MENPs are dispersed on a surface of the piezoelectric substrate.

In an embodiment of the present application, the piezoelectric substrate and the MENPs dispersed on the surface of the piezoelectric substrate form a piezoelectric layer.

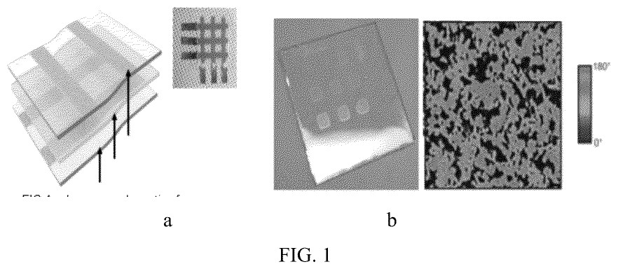

In an embodiment of the present application, a thin film flexible substrate of a three-layer composite structure is provided, as shown in FIG. 1a. It may be seen from the figure: an upper layer and a lower layer are both flexible substrates configured to provide structural support and flexibility to ensure the stability and durability of the apparatus in a complex environment; and a middle layer is the piezoelectric layer, which utilizes its piezoelectric effect to achieve the conversion between a mechanical stress and an electrical signal, thus improving the sensitivity of sensing and driving.

In an embodiment of the present application, a flexible substrate is provided, including the following steps:

Sylgard 184 (PDMS precursor) and CLA (curing agent) were mixed into a uniform solution at a mass ratio of 10:1 and annealed at 150° C. for 10 min to obtain a cured PDMS thin film; and a silver (Ag) nano particle solution with a mass fraction of 15% was then spray-coated and embedded into a PDMS substrate, and cured at 60° C. for 30 min to obtain a flexible PDMS substrate.

In the embodiment of the present application, in order to improve the mechanical resistance of the flexible substrate under various deformation conditions, an AgNW network-based nanocomposite was used as an intrinsically stretchable interconnecting material.

In an embodiment of the present application, an ethanol solution of an AgNW network (wherein a mass-volume ratio of the AgNW network to ethanol is 10 mg:1 ml) was deposited onto a polyethylene naphthalate (PEN) substrate in a spray-coating manner, and then the PEN substrate deposited with AgNW was placed on an uncured PDMS thin film, and after curing at 100° C. for 1 h, the PEN was peeled off to obtain the AgNW network embedded PDMS film. The uncured PDMS thin film was obtained by mixing Sylgard 184 with CLA in a mass ratio of 20:1 to form a uniform solution and performing annealing at 150° C. for 10 min.

Further, to facilitate the transfer of the AgNW network, a self-assembled monomolecular layer (SAM) of (1H,1H,2H,2H-perfluorooctyl) silane (FOTS) was pre-deposited on the surface of the PEN substrate as an anti-adhesion layer.

In order to further obtain a composite with excellent piezoelectric performance to meet stringent requirements of a high-performance wearable device or other related fields, the preparation of the flexible substrate was further adjusted.

In the embodiment of the present application, the flexible substrate includes, but is not limited to, a PVDF thin film, a PZT thin film, P(VDF-CTFE), etc.

In an embodiment of the present application, a flexible substrate is provided, including the following preparation steps: PVDF was dissolved in a mixed solvent of DMF (N,N-dimethylformamide) and acetone (wherein a volume ratio of the DMF to the acetone was 10:1) to obtain a PVDF dispersion with a mass fraction of 2.5%, and an AgNW network embedded PDMS film processed by UVO (ultraviolet ozone) for 50 min was coated with the PVDF dispersion to obtain the flexible substrate.

Further, a coating manner is a spin-coating method.

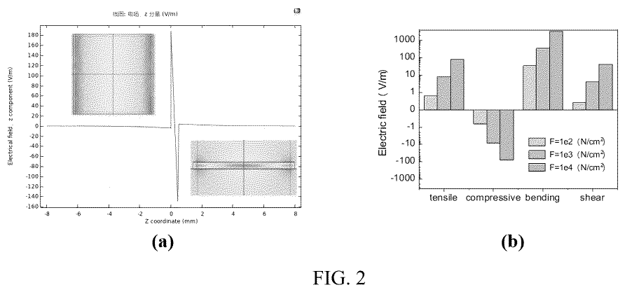

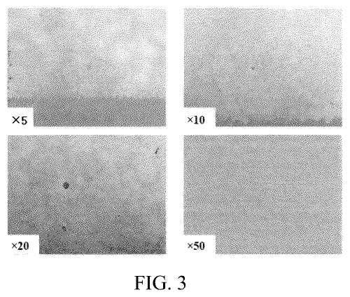

The applicant performed the following performance tests on the prepared flexible substrate (also known as “PVDF film”), including:
(1) The flexible substrate was observed and measured using a piezoelectric force microscope (PFM), and a result is shown in FIG. 1b. It may be seen from FIG. 1b: a longitudinal piezoelectric coefficient d33 of the film is between −24 pC/N and −30 pC/N.
(2) Potential and electric field distribution of the PVDF film under different strain conditions and microstructural changes after polarization processing were analyzed by experiments and simulated electric fields. Results are shown in FIG. 2 and FIG. 3.

By applying different stresses or strains to the PVDF film, the PVDF film generated different electric fields due to an inverse piezoelectric effect.

In one or some embodiments of the present application, FIG. 2a shows a relation curve of a z-direction electric field generated by the PVDF film and a z-coordinate when a small bending stress was applied to the PVDF film, and data were obtained by COMSOL simulation. Meanwhile, the potential distribution of the PVDF film from side and top views is shown through the illustration.

In one or some embodiments of the present application, FIG. 2b shows a diagram comparing electric fields generated by the PVDF film under different strain conditions. Comparison of the distribution of the electric fields generated by the PVDF film under four different stress cases shows that the intensity of the generated electric field increased with the increase of the stress amplitude.

In one or some embodiments of the present application, FIG. 3 shows ×5, ×10, ×20, and ×50 times magnified diagrams of the PVDF film after polarization processing. Control of the magnetoelectric switch was achieved by utilizing the inverse piezoelectric effect of the PVDF film. Specifically, by applying an external electric field, the PVDF film was deformed, thereby changing its internal magnetization state. Due to the antiparallel arrangement of dipoles, the α-phase PVDF film had neither ferroelectric characteristics nor piezoelectric characteristics. However, the α phase was the most readily available PVDF form. Therefore, before practical application, it was necessary to polarize it by applying a high electric field to convert it from the α phase to a β-phase to obtain desired ferroelectricity and piezoelectric characteristics.

In the embodiment of the present application, in order to control the magnetism of the MFNPs by the applied electric field (E-field), a P(VDF-TrFE) thin film capable of converting a mechanical stimulus into an electrical signal was used as a piezoelectric layer. The preparation of the P(VDF-TrFE) film includes the following steps:
a polyvinylidene fluoride-trifluoroethylene copolymer was dissolved in a mixed solvent and spread to form a film on a preprocessed flexible substrate by a spin-coating method, and the P(VDF-TrFE) thin film was obtained.

In an embodiment of the present application, the preparation of the P(VDF-TrFE) thin film includes: a PDMS substrate embedded with AgNWs was spin-coated with a methyl ethyl ketone (MEK) solution of 20 wt % P(VDF-TrFE) at a speed of 1000 rpm, and then annealing was performed under vacuum at 140° C. for 1 h to obtain the highly crystalline P(VDF-TrFE) thin film.

In the embodiment of the present application, preparation of the MENPs includes the following steps:
magnetic cores were first prepared by a hydrothermal method; and then surfaces of the magnetic cores are coated with shell layers to form core-shell structures, and the MENPs are obtained by cooling.

In an embodiment of the present application, the magnetic cores include, but are not limited to, other polymagnetic materials such as CoFe2O4 and NiFe2O4; and the shell layers are selected from ferroelectric materials such as BaTiO3, BiFeO3, and PZT(Pb(Zr,Ti)O3).

In an embodiment of the present application, preparation of the MENPs includes the following steps:
the CoFe₂O₄ magnetic cores were first prepared by a hydrothermal method; and then surfaces of the CoFe₂O₄ magnetic cores were coated with BaTiO₃ shell layers to form core-shell structures, and the MENPs were obtained by cooling.

In an embodiment of the present invention, by reducing the cooling rate, i.e., from above 52° C./min to below 14° C./min using a CMF1100 control furnace, an average diameter of the MENPs may be controlled from less than 25 nm to greater than 100 nm, and particle size distribution is controlled to within 30%. The particle size distribution was measured using a ZetasizerNano series utilizing a standard dynamic light scattering (DLS) method.

Further, the cooling rate was 14-52° C./min, and the average particle size distribution range of the MENPs was 19-31 nm.

In an embodiment of the present application, CoFe₂O₄ particles are prepared by dissolving an aqueous solution of a soluble cobalt salt, a soluble iron salt, and polyvinylpyrrolidone in an aqueous solution of sodium borohydride, and performing heating and mixing.

A BaTiO₃ precursor solution is prepared by mixing an aqueous solution containing barium carbonate and a citric acid with an ethanol solution containing titanium isopropoxide and a citric acid.

The MENPs are obtained by dispersing the CoFe₂O₄ particles in the BaTiO₃ precursor solution, performing ultrasonic processing, drying, and calcination, and then controlling the cooling rate.

Further, the preparation of the CoFe₂O₄ nano particles includes: an aqueous solution (15 mL) of 0.058 g of Co(NO₃)₂·6H₂O, 0.16 g of Fe(NO₃)₃·9H₂O, and 0.2 g of polyvinylpyrrolidone (PVP) was dissolved in 5 mL of an aqueous solution containing 0.9 g of sodium borohydride (NaBH₄), and a reaction was performed for 12 h at 120° C. to obtain the CoFe₂O₄ nano particles.

Further, the preparation of the BaTiO₃ precursor solution includes: 30 mL of an aqueous solution containing 0.029 g of barium carbonate and 0.1 g of citric acid was mixed with 30 mL of an ethanol solution containing 0.048 mL of titanium isopropoxide (Ti(OiPr)) and 1 g of citric acid to obtain the BaTiO₃ precursor solution.

Further, the preparation of the MENPs of the CoFe₂O₄@BaTiO₃ core-shell structure includes: 0.1 g of CoFe₂O₄ nano particles were dispersed in the BaTiO₃ precursor solution, and a mixture was ultrasonically dispersed for 2 h, then dried at 60° C. with continuous stirring overnight, then calcined at 780° C. for 5 h, and cooled at a rate of 14° C./min, and the MENPs with an average particle size distribution range of about 19-31 nm are obtained.

To validate the design and performance of the flexible magnetoelectric piezoelectric switch apparatus and to detail a working mechanism, fabrication process and characteristics of the apparatus, in the embodiment of the present application, in order to utilize the unique magnetoelectric coupling effect of these particles, thereby enhancing or imparting new functional characteristics to the material, MENPs are deposited in a piezoelectric substrate, i.e., a P(VDF-TrFE) thin film.

In an embodiment of the present application, dispersing the MENPs in the piezoelectric layer specifically includes:
an MENPS solution (made by dispersing CoFe₂O₄ particles in a BaTiO₃ precursor solution) was deposited on a surface of the P(VDF-TrFE) thin film by a drop-coating method.

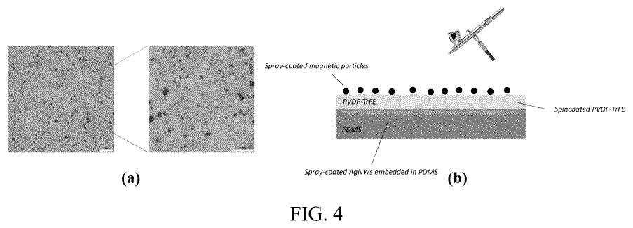

In order to fully disperse the magnetic nano particles, further, the magnetic particles were uniformly deposited onto the surface of the P(VDF-TrFE) thin film by a spray-coating method, and the MENPs solution was diluted with ethanol at a volume ratio of 1:10. An image of the magnetic nano particles deposited on the surface of the P(VDF-TrFE) thin film is shown in FIG. 4a. At the same time, an entire preparation process flow is described in detail by means of 4b.

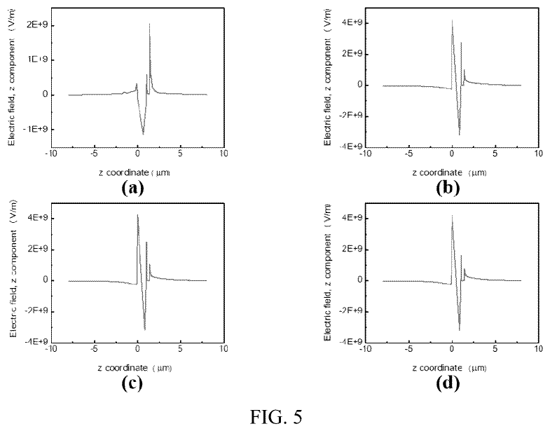

In an embodiment of the present application, FIG. 5 shows a diagram of changes in electric field distribution at different distances between composite NPs and a PVDF film, including (A) 0 nm, (B) 10 nm, (C) 20 nm, and (D) 40 nm. An external electric field generated outside the PVDF film decreased as the distance increased. From the figure, there is a tendency for the electric field around the NPs to weaken as the distance increased, and this change led to a decrease in a magnetization intensity. Therefore, a magnetization state of the NPs may be effectively controlled by adjusting the distance between the NPs and the PVDF film.

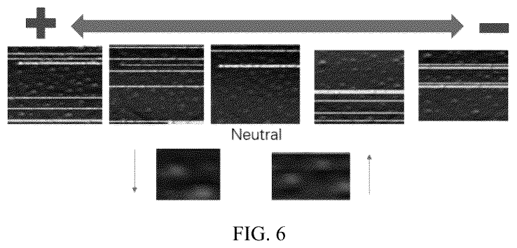

In an embodiment of the present application, FIG. 6 shows an image of NPs based on a magneto-optical Kerr effect (MOKE), reflecting a change in the magnetization state of the NPs as a stress applied to the piezoelectric substrate increased.

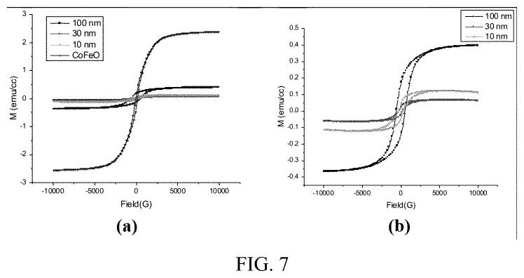

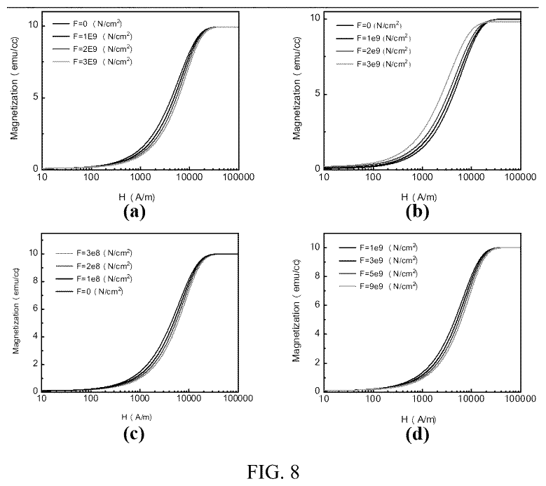

In an embodiment of the present application, FIGS. 7 (a) and 7 (b) show M-H hysteresis loops of CoFe₂O₄ present in forms of NPs and a thin film respectively. The saturation magnetization intensity of the CoFe₂O₄ thin film was higher compared to nano particles. Furthermore, FIG. 7 (b) shows that CoFe₂O₄ had a highest saturation magnetization intensity when the size of the nano particles was 100 nm, whereas the saturation magnetization intensity decreased significantly when the size was reduced to 10 nm.

In an embodiment of the present application, FIGS. 8a-d show M-H hysteresis loops obtained under different stress conditions. First, a vertically upward magnetic field was applied in a simulation model and made to scan from 0 A/m to 105 A/m, and four stress cases (tensile, compressive, bending, and shear) were simulated respectively.

Stresses of different magnitudes significantly affected the M-H hysteresis loops of the magnetic nano particles (NPs). As the applied stress increased, the M-H curves under the tensile, bending and shear cases gradually became smoother, whereas the M-H curve under the compressive case became steeper. A reason for this phenomenon may be observed in FIG. 8b, where it is shown that a direction of the electric field generated under the compressive case was opposite to that of other cases. Under the tensile, bending and shear cases, the direction of the electric field generated by the PVDF film was almost parallel to the direction of the applied magnetic field, and therefore, an electrostriction effect of the ferroelectric shell layer led to a shape change of the core magnetic nano particles, which facilitated the magnetization process. In contrast, under the compressive case, the effect prevented the magnetization from occurring.

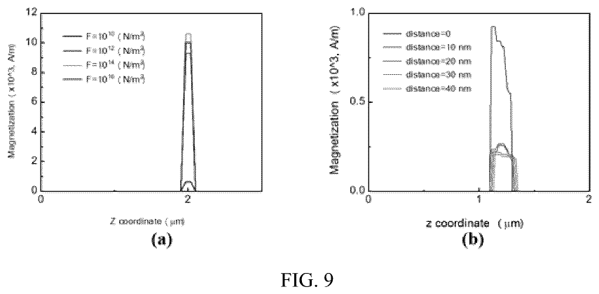

In an embodiment of the present application, FIGS. 9a and 9b respectively show (a) a change curve of the magnetization intensity with respect to a Z coordinate at different stress amplitudes, and (b) a distance (the distance here refers to spacing between the PVDF substrate and centers of the composite nano particles) dependence curve for the change of the magnetization intensity with respect to the Z coordinate. As shown in FIG. 8B and FIG. 2, the electric field generated by the bending stress was stronger compared to other stress cases under the same stress conditions, and thus the magnetization state could be effectively changed even if a smaller stress is applied. Therefore, applying a bending stress was a more efficient manner of switching the magnetization and will be explained in detail as an example in the subsequent analysis of simulation results.

FIG. 9a illustrates that as the stress amplitude increased, the magnetization intensity increased accordingly and eventually reached the saturation magnetization intensity. Based on this, a stress-induced magnetoelectric switch may be implemented.

FIG. 9b illustrates that as the distance increased, the magnetization intensity decreased accordingly. A main reason for this may be derived from the diagrams of electric field distribution at (A) 0 nm, (B) 10 nm, (C) 20 nm and (D) 40 nm in FIG. 5.

In the present application, in order to manipulate the magnetism of the MF nano particles through the generated electric field, a P(VDF-TrFE) thin film was used to achieve the conversion of a mechanical stimulus to electrical energy.

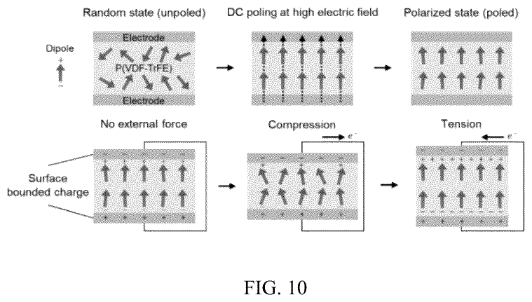

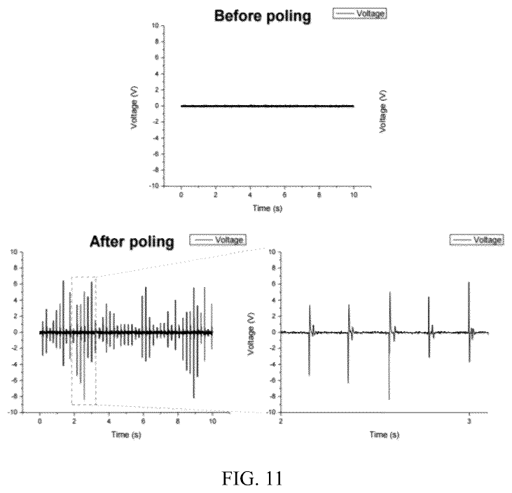

In one or some embodiments of the present application, as shown in FIG. 10, polarization phenomena of a flexible substrate under different stress conditions are provided.

In one or some embodiments of the present application, as shown in FIG. 11, voltage changes during a polarization process of a flexible substrate are provided.

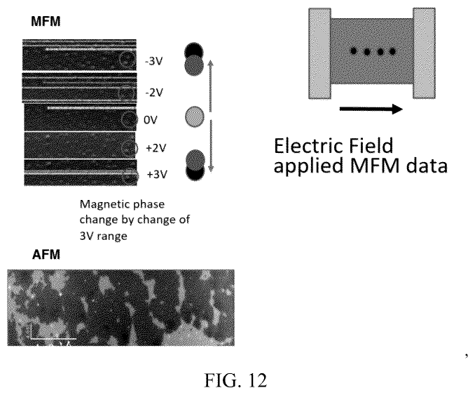

In one or some embodiments of the present application, as shown in FIG. 12, characteristics of a magnetic phase change of a flexible substrate are provided.

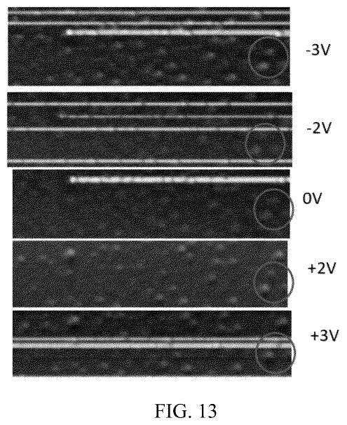

Further, FIG. 13 shows responses of MENPs to voltage changes.

In the present application, in order to achieve wearable characteristics and controllable functions, the flexibility and inverse electric effect of the P(VDF-TrFE) thin film were utilized, so that a high-quality flexible-phase P(VDF-TrFE) thin film could be fabricated to form a piezoelectric substrate.

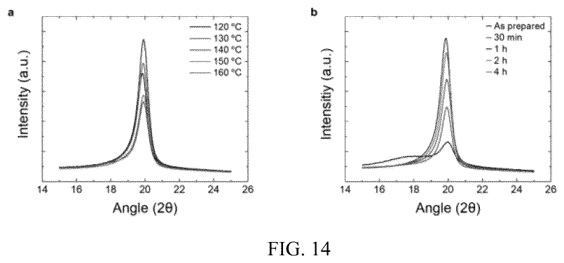

In one or some embodiments of the present application, XRD measurement results of a P(VDF-TrFE) film at different sintering temperatures (A) and sintering times (B) are provided, as shown in FIG. 14. It may be seen from the figure that optimized sintering conditions are 140° C. and a sintering time of 1 hour.

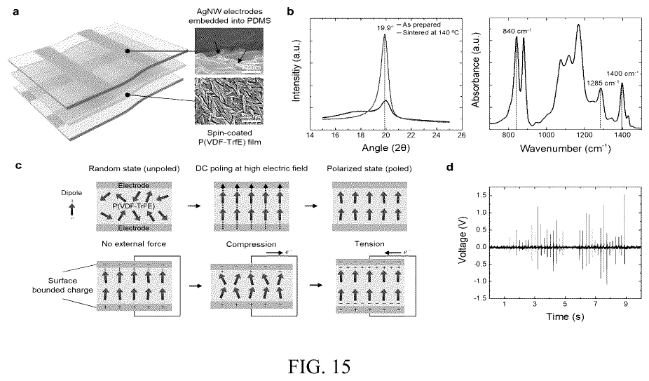

In an embodiment of the present application, a conceptual diagram of a soft piezoelectric device composed of an AgNW electrode and a P(VDF-TrFE) thin film is provided, as shown in FIG. 15a. It may be seen from the figure that an AgNW network in an AgNW network embedded PDMS substrate is well dispersed, and the P(VDF-TrFE) thin film is well crystallized.

FIG. 15b shows results of X-ray diffraction (XRD) and Fourier transform infrared (FTIR) spectral analysis of P(VDF-TrFE). A β-phase crystal structure of PVDF was confirmed by an XRD spectrum based on peaks at different sintering time conditions, which was attributed to a peak at 2θ=20.26° related to the diffraction of a β phase at (110) and (200). An FTIR spectrum showed vibrational bands at 840 cm⁻¹, 1279 cm⁻¹, and 1400 cm⁻¹, further verifying the existence of such a crystal structure, and indicating that the P(VDF-TrFE) substrate was capable of generating the electric field uniformly.

To further activate a piezoelectric material, a direct current (DC) polarization process was performed on the P(VDF-TrFE) substrate by applying a direct current voltage, so as to align surface bounded charges. The process is shown in FIG. 15c, which provides a schematic diagram of a high-voltage polarization process and its piezoelectric operation under mechanical deformation, explaining how the surface bounded charges could be aligned by applying the direct current voltage, so as to activate the piezoelectric material, including: when a mechanical stimulus is applied to the piezoelectric device, electrons at the surface move on an electrical load, generating an electrical signal.

FIG. 15d shows the piezoelectric performance of a polarized piezoelectric device. Here it may be seen that the device was capable of generating a voltage output of 0.1 to 1.5 V under a mechanical stimulus, demonstrating its effective energy conversion capability.

In order to study multifunctional nano particles (NPs) on a wearable surface, the optimization of spacing of the nano particles and their optimal responses under a strain, an electric field, or magnetization were studied. In this patch, strain-regulated magnetization was a key factor. In other words, electric, magnetic and elastic fields needed to achieve coupling with a minimum energy loss. Between the magnetostriction effect and the piezoelectric effect, the magnetoelectric effect (ME effect) of nano particles of the core/shell structure was achieved by applying an external strain. A piezo-element included a P(VDF-TrFE) thin film and BTO (barium titanate) and followed a linear strain-charge intrinsic relationship. In addition, in order to accurately describe the magnetization state characteristics of a magnetostrictive material, a nonlinear isotropic model was used for CFO (cobalt ferrite).

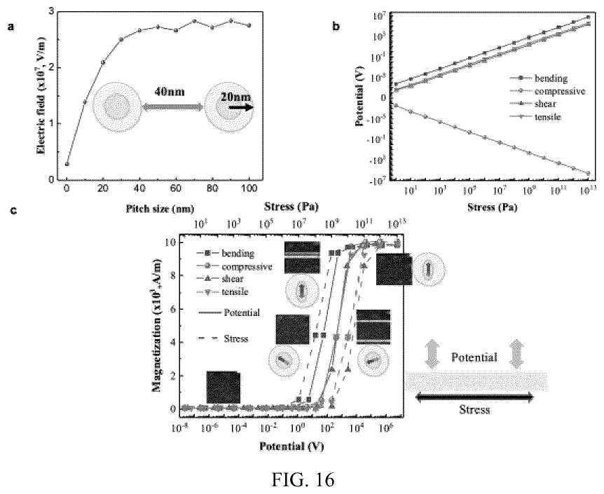

In an embodiment of the present application, an MENP device is provided, physical characteristics of which were tested and analyzed as follows, and spacing optimization was tested to avoid possible interferences to check the above effects. The test and analysis results are shown in FIG. 16.

In an embodiment of the present application, when a stress was applied to the P(VDF-TrFE) thin film, the change in the electric field of the nano particles with a diameter of 20 nm at different nano particle spacing is shown in FIG. 16a. It may be seen from the figure that a potential of the electric field first increased and then tended to saturate with the increase of the particle spacing. This indicated that optimizing the spacing between MENPs is essential to avoid possible interference and maximize the electric field effect.

In an embodiment of the present application, different types of stresses were applied to the P(VDF-TrFE) thin film, and four deformation modes were analyzed: bending, compressive, tensile, and shear stresses by generating a magnetized electric field through the P(VDF-TrFE) thin film. The results are shown in FIG. 16b. FIG. 16b shows results of calculation simulations of potential changes generated by different types of stresses (including bending, compressive, shear, and tensile deformation modes). An M-E relationship from the P(VDF-TrFE) thin film to CFO nano particles was studied. A nano particle distribution density needed to be high enough to induce magnetization, which provides a technical basis for the reliability of a wearable patch.

In an embodiment of the present application, a relationship between the magnetization of MENPs on the patch and the potential was analyzed, as shown in FIG. 16c, where particular attention was paid to the change of the magnetization state of cobalt ferrite (CFO) nano particles with respect to the potential and stress on the PVDF substrate. The figure also characterizes the corresponding magnetization states at different stress levels by a low-moment magnetic force microscope (MFM), illustrating a conversion mechanism from stress to potential and then to magnetization.

Combined with the results of the deformation modes shown in FIG. 16b and FIG. 16c, according to the MF curves, the potential generated at the same stress level could overcome a threshold voltage and thus reach saturation magnetization faster.

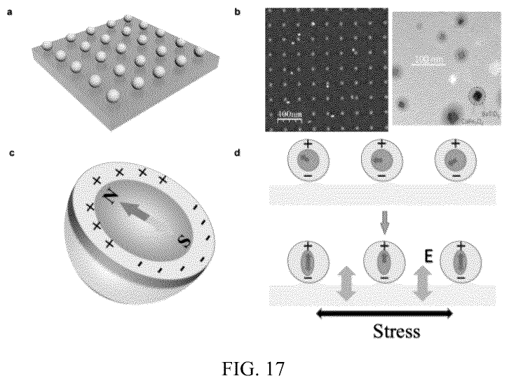

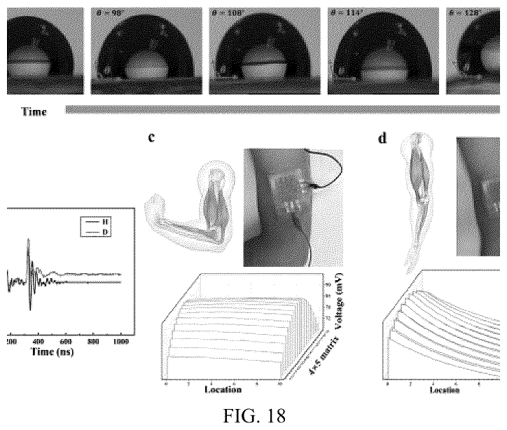

In one or some embodiments of the present application, MENPs were deposited onto the surface of a P(VDF-TrFE) thin film applied to a flexible substrate, and the MENPs were aligned by applying a demagnetization field, as shown in FIGS. 17 and 18.

In an embodiment of the present application, a controllable ME nanopatch is provided, as shown in FIG. 17a. A PVDF film was used as a substrate to control the magnetism of MF nano particles. Through structural optimization, P(VDF-TrFE) was used to control the magnetism of the MENPs deposited thereon, wherein the nano particles could be aligned by applying a demagnetization field. An MF nanocomposite thin film was prepared in which BTO-CFO nano particles were uniformly dispersed in a PVDF polymer.

Dynamic changes of MENPs in the above steps were analyzed using an atomic force microscope (AFM) and a transmission electron microscope (TEM), and results are shown in FIG. 17b. It may be seen from the figure that MENPs could be aligned by manipulating the demagnetization field. An AFM image on the right shows that by controlling the trend of the demagnetization field to adjust a coffee ring effect, a neat alignment of MENPs may be achieved. A TEM image on the right shows a composite structure of the MENPs, which is about 20 nm in size. An inner core is CFO, an FM (ferromagnetic) material, and an outer shell is BTO, an FE material. MENPs of a core-shell composite structure were used as MENPs due to a high magnetoelectric coupling coefficient.

In an embodiment of the present application, a CFO@BTO nano particle of a core/shell structure is provided, as shown in FIG. 17c. Due to the magnetoelectric effect, the particle could convert magnetization to a potential, and conversely, the BTO shell could convert a potential to magnetization. FIG. 17c provides ideal characteristics of the CFO@BTO nano particles: inside the MENPs, the ferromagnetic CFO particles are in a state of synchronized orientation of atomic magnetic moments. However, due to the presence of the outer BTO shell, it was polarized under the action of an applied electric field and generated opposite charges at two ends of the nano particle shell layer. This magnetoelectric effect (ME effect) enabled the nano particles to achieve the conversion from magnetization to a potential; and conversely, the BTO layer was also able to convert the electric field to magnetization.

The P(VDF-TrFE) film was used as a piezoelectric substrate to control the magnetic characteristics of MENPs deposited on its surface, wherein the nano particles could be arranged by applying a demagnetization field. Nano particles of the composite core-shell structure were used for the MENPs due to their high magnetoelectric coupling coefficient.

FIG. 17d shows a schematic diagram of a multiferroic mechanism in this case. By applying a force, the potential change in P(VDF-TrFE) and the BTO shell led to a change in the magnetization in the CFO core. Red/blue arrows indicate changes in the direction of the electric field and magnetization. After a strain was applied, an electric field was generated to align the BTO shell. The direction of magnetization was then controlled. FIG. 17d shows a final MENP patch function after nano particles (NPs), P(VDF-TrFE), and nano particles deposited on the P(VDF-TrFE) were optimized: this piezoelectric magnetoelectric switch did not respond in the absence of a mechanical load, i.e., a magnetic moment was randomly oriented, as shown at the top of FIG. 17d. When a stress was applied to the PVDF (a black arrow at the bottom of FIG. 17d), a potential was generated on its exterior (a green arrow), thus activating the BTO shell layers of the MENPs to deform due to the electrostriction effect. This strain was further transferred to the internal CFO particles, so that the directions of the magnetic moments converged due to the magnetostriction effect. The red/blue arrows indicate changes in the direction of magnetization (upward and downward direction/north-south direction) respectively.

After the fabrication process was successfully optimized, we tested an overall device function of the MENP patch.

FIGS. 18a-m show characteristics and performance of an MENP patch.

FIGS. 18a-e show an experiment on hydrophobicity of the patch and its analysis of changes over time (from left to right). Over time, an angle between a water droplet and a surface contact line increased from 95 degrees to 128 degrees. For a wearable device, hydrophobicity is essential to improve surface reliability and may effectively protect the device from an aqueous solution. In addition, this characteristic makes the device washable, thus improving the usability of the MENP patch in practical applications. It is noteworthy that the contact angle increased with time (from 95 degrees in FIG. 18a to 128 degrees in FIG. 18e).

In an embodiment of the present application, a magnetic field sensing MENP device integrated with an array of miniature Hall sensors is adopted, as shown in FIG. 18f. A generated magnetic field was observed in real time by the mounted array of miniature Hall sensors to confirm a magnetization output. The sensors were arranged in a 3×3 matrix in an MENP region, as shown in FIG. 18f. A magnetic signal induced by a human motion was transmitted to the MENPs through PVDF and sensed by the change in the electric field between the MENPs and the sensors.

FIG. 18g shows a piezoelectric echo signal after pulse generation, demonstrating the performance of the MENP patch in terms of electric field-driven magnetization. By applying an alternating magnetic field pulse, a corresponding electric field response could be observed. First and second magnetic echo signals after the generation of the piezoelectric effect may be seen in FIG. 18g. This result confirmed that an interface between the MENPs and the piezoelectric substrate was well connected and functioned properly. A generated potential led to magnetization as shown in FIG. 18c. In this configuration, an opposite conversion of magnetization to an electric field was also observed. A received electrical signal was attributed to an elastic deformation of P(VDF-TrFE), and the deformation was caused by uniform magnetization of the particles.

FIG. 18h shows a spin-echo signal after a transducer pulse.

In order to study signal changes during wearing, we performed wearable motion tests by fixing the MENP patch on a human body. The response of the magnetic field induced by the human body motion was observed, and relevant results are shown in FIG. 18i.

FIG. 18i and FIG. 18j show the action of an elbow joint in flexion and extension modes and response characteristics of the patch respectively. In FIG. 18i, the elbow joint was flexed to drive the forearm toward the shoulder until the hand was flush with the shoulder, a voltage response of the patch under changes in the magnetic field is shown, and an electric field range of about 120 A/m was observed. FIG. 18j provides a detailed schematic diagram of a mannequin and gait analysis, with an estimated force of approximately 75 N based on the human motion. The experimental test results were highly consistent with the simulation analysis.

FIGS. 18k-18m show measurement results of magnetic field responses induced by the human motion. The distribution of the magnetic field responses was observed in different modes using the patch fixed to the triceps brachii muscle. The test results for contraction (low tension) to relaxation (high tension) are shown in FIG. 18k (low tension), FIG. 181 (medium tension), and FIG. 18m (high tension) respectively. The magnetic field responses indicated that the human motion magnetoelectric sensor operated stably and reliably.

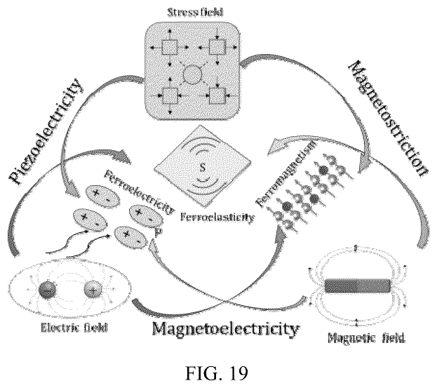

In an embodiment of the present application, a spin nanopatch is provided, as shown in FIG. 19.

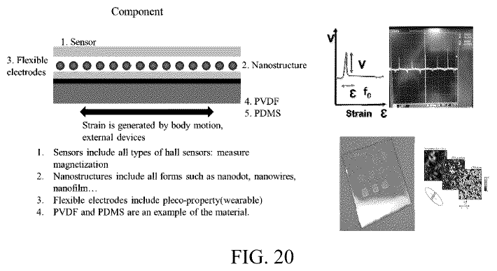

In an embodiment of the present application, constituent components of a nanopatch are shown in FIG. 20.

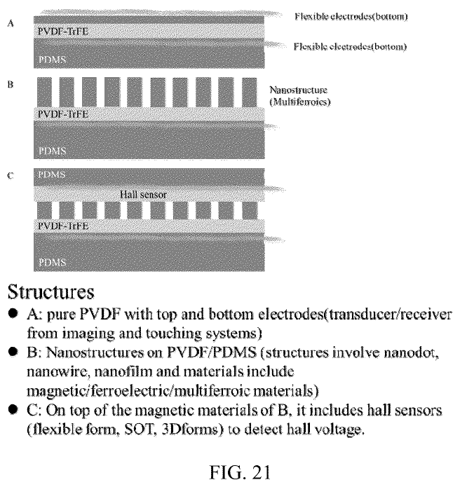

In some embodiments of the present application, three nanopatch structures are shown in FIG. 21.

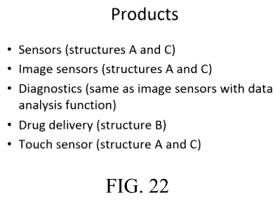

In some embodiments of the present application, exemplary products of a nanopatch are shown in FIG. 22.

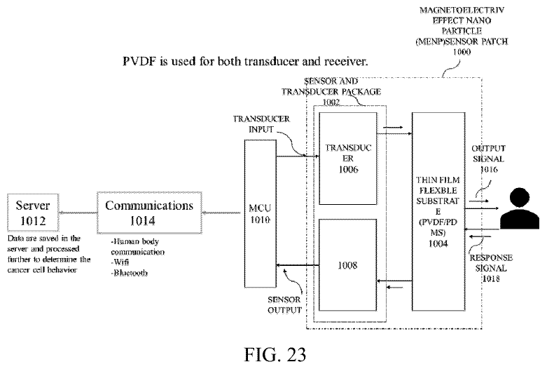

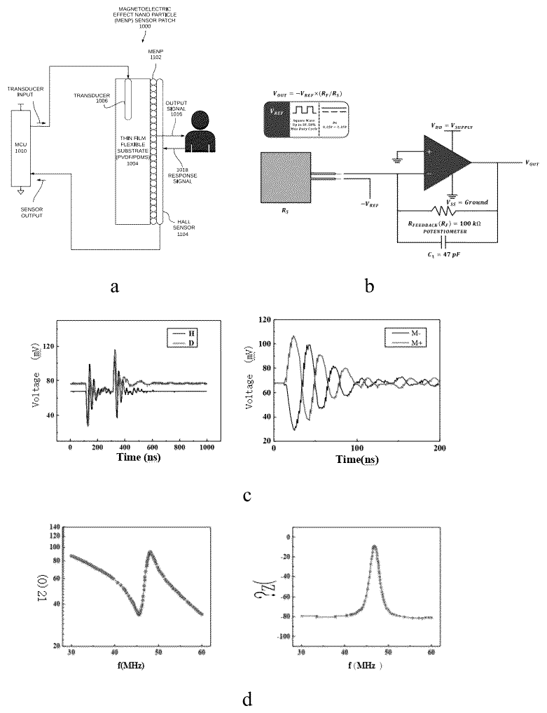

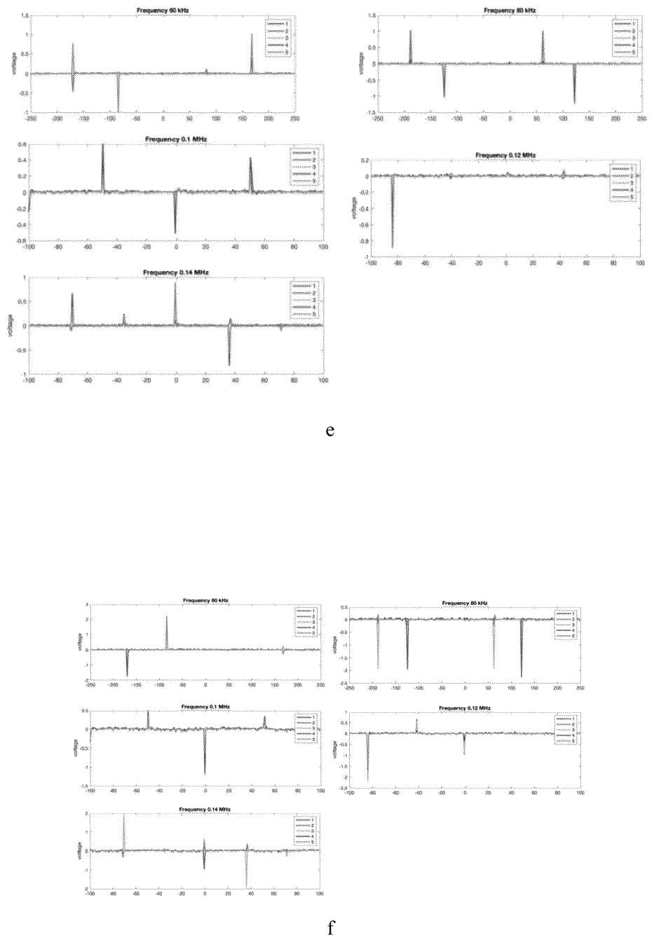

In an embodiment of the present application, as shown in FIG. 23, a system is provided, wherein PVDF is used as both a transceiver and a receiver.

Further, a magnetoelectric effect nano particle (MENP) sensing patch 1000 includes a sensor and transducer assembly 1002 and a thin film substrate PVDF/PDMS 1004. The sensor and transducer assembly 1002 includes a transducer 1006 and a sensor 1008.

During operation, a microcontroller (MCU) 1010 transmits an input signal to the transducer 1006, and the transducer 1006 generates a transducer output signal and inputs it to the thin film substrate (PVDF/PDMS) 1004, resulting in an output signal which is subsequently received by the tissue of a user.

The tissue of the user generates a response signal 1018 that is returned to the thin film substrate (PVDF/PDMS) 1004, which in turn forms a sensing input signal that is input to the sensor 1008. The sensor 1008 generates a sensing output signal and returns it to the MCU 1010.

The MCU 1010 transmits the sensing output signal to a server 1012 via a communication interface 1014. The communication interface 1014 utilizes Wi-Fi or Bluetooth communication based on human body communication. The server 1012 is responsible for storing data and further processing the data to analyze a behavior of a cancer cell.

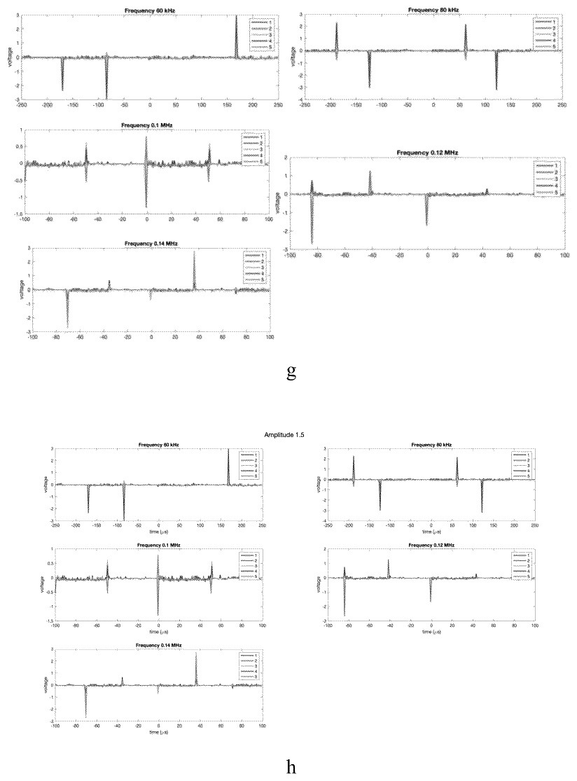

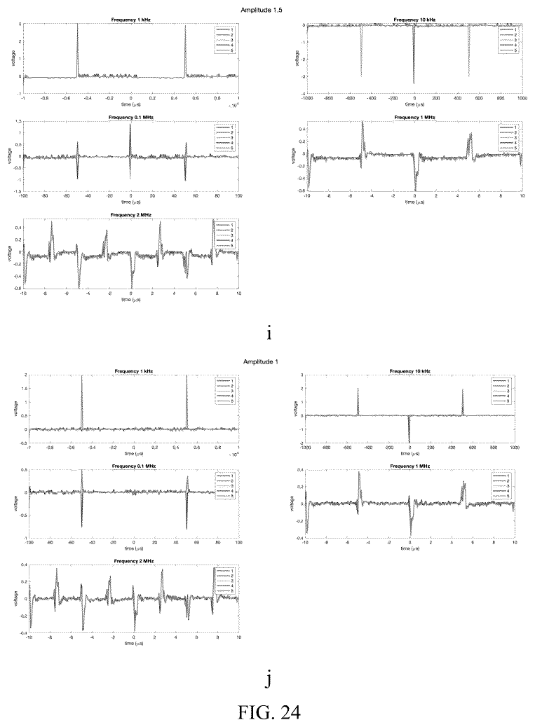

In an embodiment of the present application, a system containing a magnetoelectric effect nano particle (MENP) sensing patch 1000 (shown in FIG. 23) is shown in FIG. 24a. The patch 1000 is composed of a substrate 1004, an MENP 1102, a transducer 1006, an MENP 1102, and a Hall sensor 1104. The transducer 1006 is connected to a thin film flexible substrate 1004, receives a transducer input signal from the MCU 1010, and subsequently generates a transducer output signal 1016 which is transmitted to the tissue of the user. In an embodiment, the transducer input signal may be a sine wave, square wave, or other waveform signal with a particular peak level.

A response signal 1018 of the tissue of the user is received by the Hall effect sensor 1104, and the output signal generated by the sensor is transmitted to the MCU 1010. A Hall voltage is obtained from the signal measurement of the MENPs.

Further, the MCU 1010 generates a digital signal for detecting a specific tissue condition by detecting a voltage difference between the transducer input signal and the sensor output signal.

In an embodiment of the present application, a circuit for generating an output signal 1016 is shown in FIG. 24b. The circuit illustrates signal waves shown in FIG. 24c-d.

FIGS. 24e-j show waveforms of the signals generated and received in the system shown in FIG. 24a. In different embodiments, the transducer signal may be a sine wave, square wave, and/or monopulse signal.

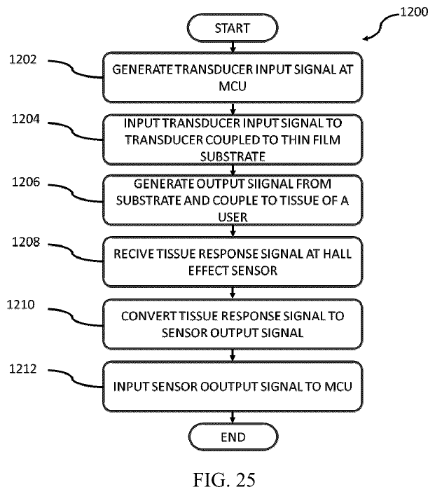

In an embodiment of the present application, as shown in FIG. 25: a method 1200 for operating a magnetoelectric effect nano particle (MENP) sensing patch 1000 is shown (as shown in FIG. 24), including:
step 1202: a transducer signal is generated at an MCU device.
Step 1204: the transducer input signal is input to a transducer coupled to a thin film flexible substrate.
Step 1206: the transducer receives the transducer input signal and generates an output signal which is transmitted through the thin film flexible substrate to the tissue of the user.
Step 1208: a Hall effect sensor receives a response signal from the tissue of the user, the sensor being connected to the thin film flexible substrate.
Step 1210: the Hall effect sensor converts the tissue response signal into a sensor output signal.
Step 1212: the sensor inputs the output signal to the MCU.

Thus, the method 1200 enables the magnetoelectric effect nano particle sensing patch 1000 to provide the transducer signal to the tissue of the user and to process the tissue response signal. It should be noted that the operational flow of the method 1200 is only an example, and specific steps may be reordered, added, subtracted, or modified according to implementation needs.

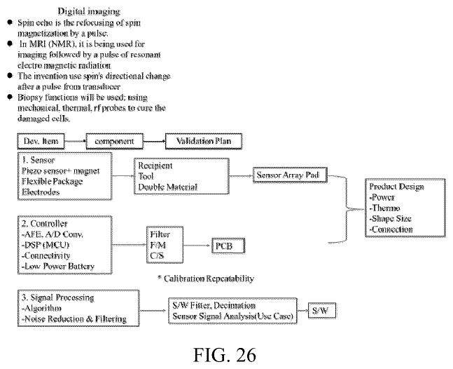

In an embodiment of the present application, various functional capabilities of a system containing a nano particle sensing patch are shown in FIG. 26.

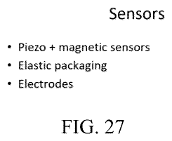

In an embodiment of the present application, relevant characteristics of the nano particle sensing patch are shown in FIG. 27.

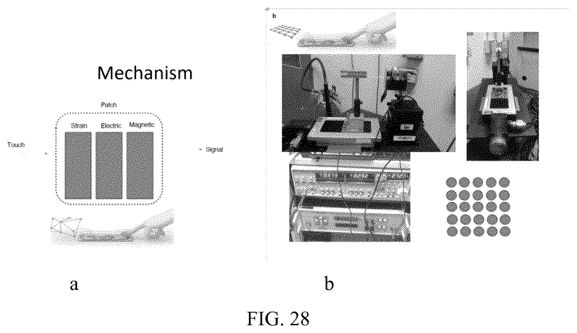

In an embodiment of the present application, a working mechanism of a nano particle sensing patch is shown in FIG. 28a.

In an embodiment of the present application, voltage measurement results of a Hall effect sensor for a mechanism of a nano particle sensing patch are shown in FIG. 28b.

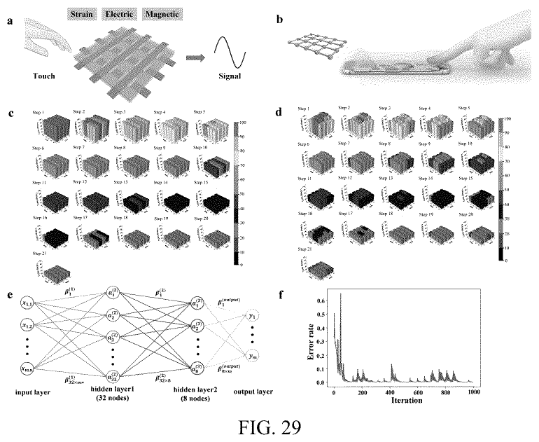

In an embodiment of the present application, a schematic diagram of a working mechanism of a touch sensor is shown in FIG. 29a, illustrating a process from touch to signal reading.

In an embodiment of the present application, a schematic diagram of matrix measurement characteristics is shown in FIG. 29b, illustrating the signal change upon touch. The number of matrices may be increased as required, and the inclusion of a plurality of matrices enables detection of different shapes and materials (hardness) and ensures ultra-high sensitivity touch detection.

In an embodiment of the present application, a method for measuring a planar surface is shown in FIG. 29c.

In an embodiment of the present application, a method for measuring a curved surface is shown in FIG. 29d.

In an embodiment of the present application, a schematic diagram of a machine learning algorithm for determining a stopping point is shown in FIG. 29e.

In an embodiment of the present application, a method for achieving zero-error statistical analysis using machine learning is shown in FIG. 29f.

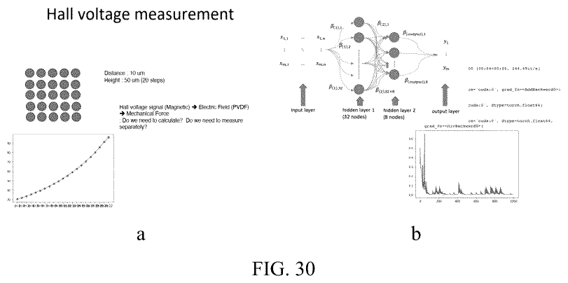

In some embodiments of the present application, structural characteristics of different layers of a nano particle sensing patch are shown in FIG. 30.

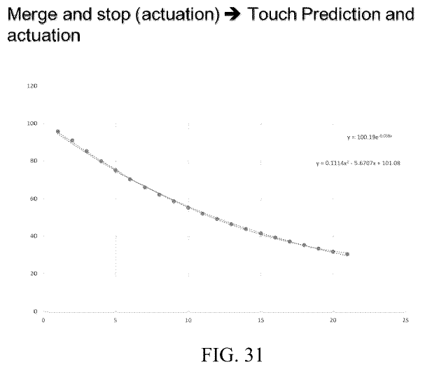

In an embodiment of the present application, a sensor actuation mechanism for a nano particle sensing patch is shown in FIG. 31.

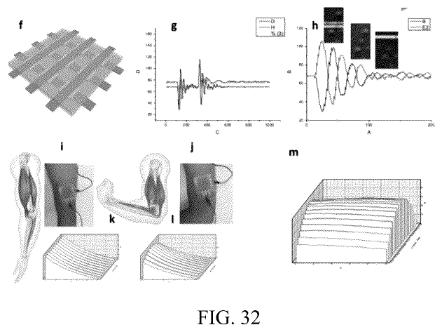

In one or some embodiments of the present application, a nano particle sensing patch and various application positions on human tissue are shown in FIG. 32.

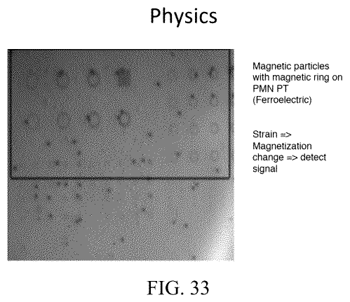

In an embodiment of the present application, detection characteristics of a nano particle sensing patch are shown in FIG. 33, illustrating that a strain leads to changes in magnetization.

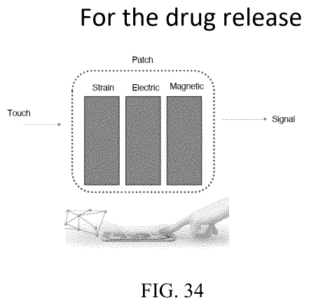

In an embodiment of the present application, a nano particle sensing patch for a drug release application is shown in FIG. 34.

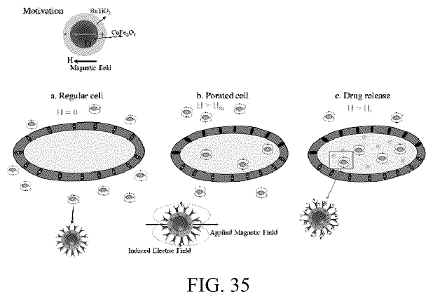

Further, FIG. 35 illustrates the characteristics of the nano particle sensing patch during drug release.

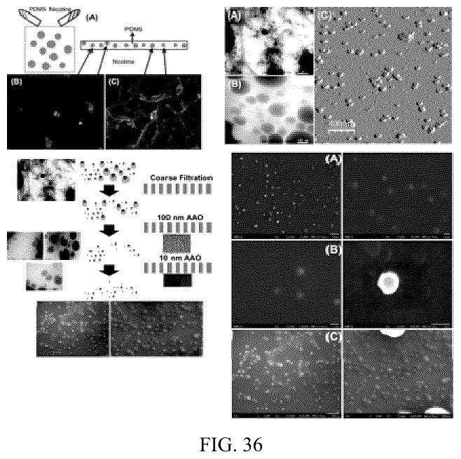

In an embodiment of the present application, characteristics of a nano particle sensing patch during nicotine drug release are shown in FIG. 36.

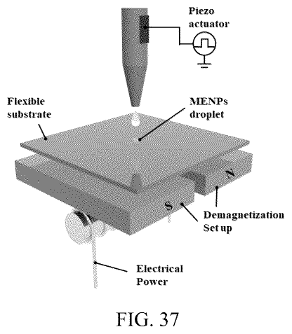

In an embodiment of the present application, as shown in FIG. 37, a method for depositing MENP droplets on a flexible substrate during an inkjet printing operation is provided.

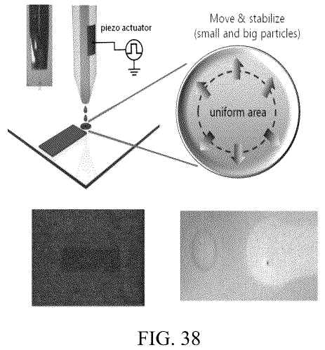

In an embodiment of the present application, as shown in FIG. 38, a method for an inkjet printing operation for depositing MENP droplets on a flexible substrate is provided.

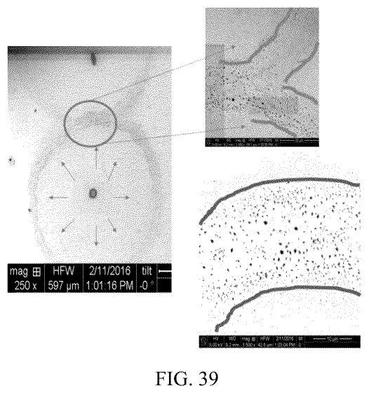

In an embodiment of the present application, as shown in FIG. 39, detailed characteristics of an MENP droplet deposition formed during an inkjet printing process are provided.

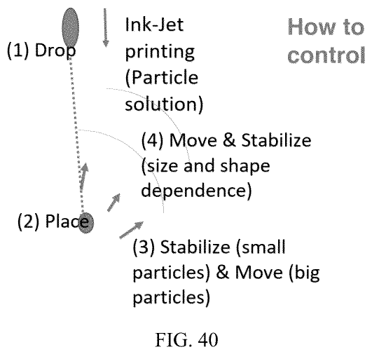

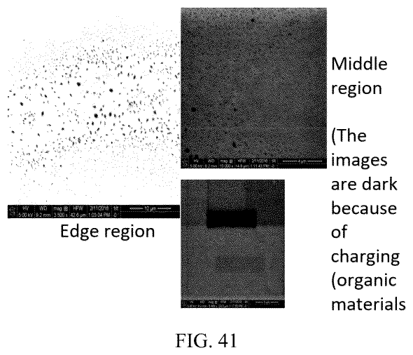

In an embodiment of the present application, as shown in FIG. 40, a manner of placing and moving droplets of a particle solution is provided. Further, as shown in FIG. 41, an edge region of a droplet is shown.

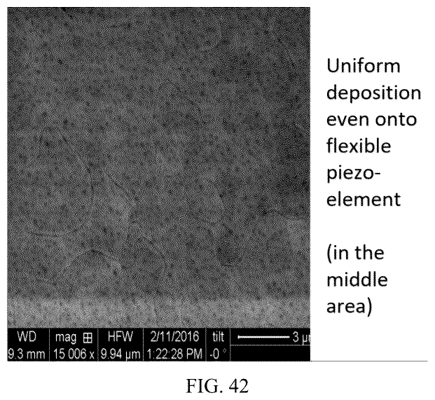

In an embodiment of the present application, as shown in FIG. 42, a method for uniform droplet deposition on a flexible piezo-element is provided.

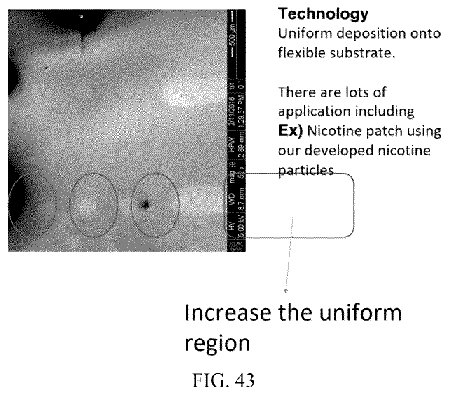

Further, FIG. 43 illustrates uniform droplet deposition on a flexible substrate.

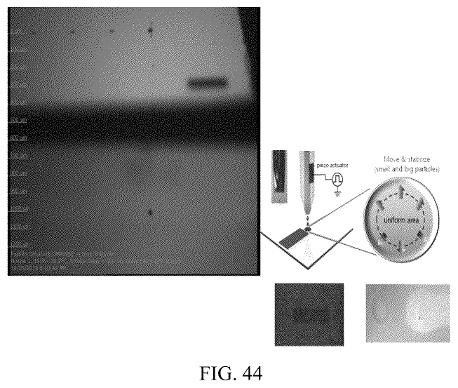

Further, FIG. 44 illustrates a manner in which small and big particles are moved and stabilized.

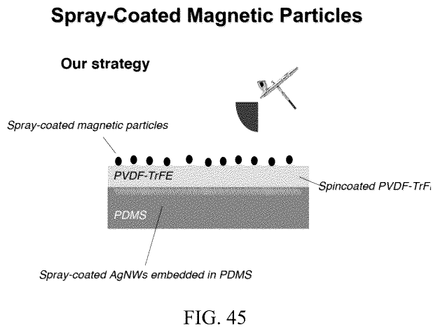

In an embodiment of the present application, as shown in FIG. 45, a method of spray-coating magnetic particles is provided.

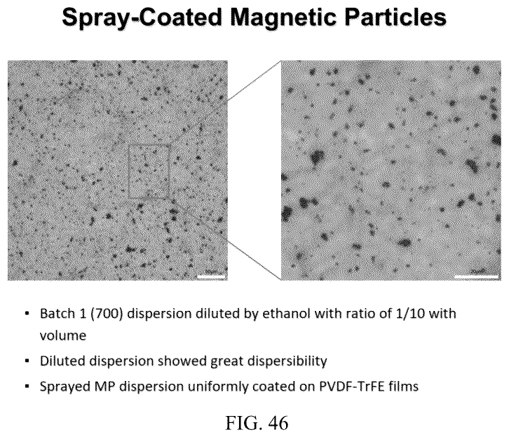

Further, FIG. 46 illustrates a detailed view of spray-coated magnetic particles.

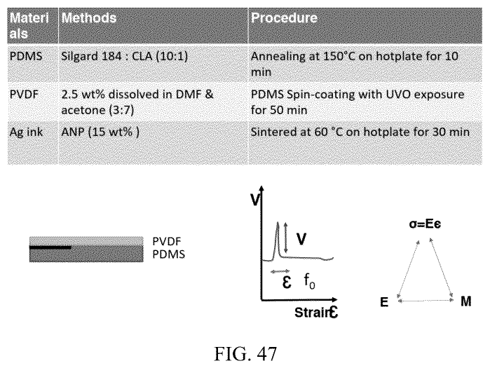

In one or some embodiments of the present application, as shown in FIG. 47, a number of methods and processes for forming a nano particle sensing patch are provided.

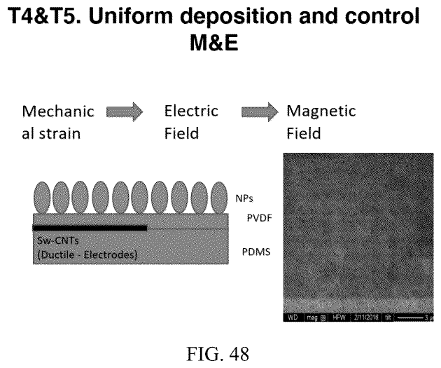

In one or some embodiments of the present application, as shown in FIG. 48, a method for how particles are uniformly deposited so that a mechanical strain induces a change in a magnetic field is provided.

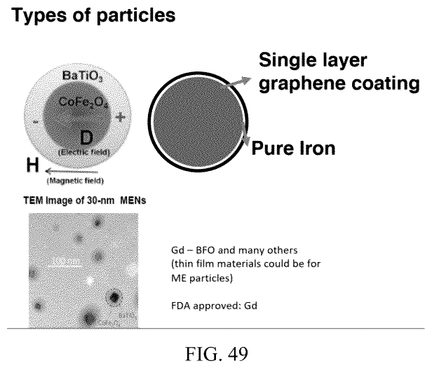

In one or some embodiments of the present application, as shown in FIG. 49, exemplary particle types are provided.

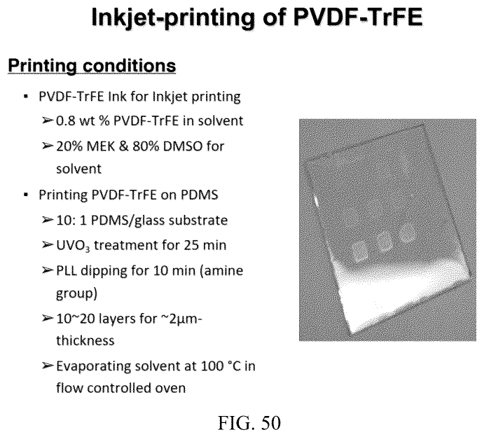

In an embodiment of the present application, as shown in FIG. 50, a method for inkjet printing of P(VDF-TrFE) is provided.

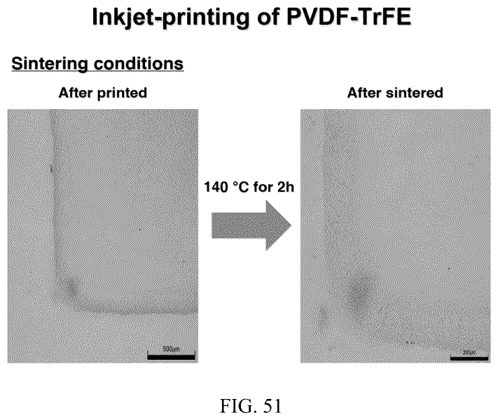

Further, FIG. 51 illustrates sintering conditions for inkjet printing of P(VDF-TrFE).

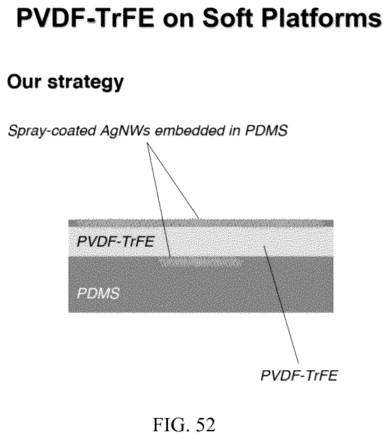

In an embodiment of the present application, as shown in FIG. 52, a scenario of performing P(VDF-TrFE) inkjet printing on a soft platform is provided.

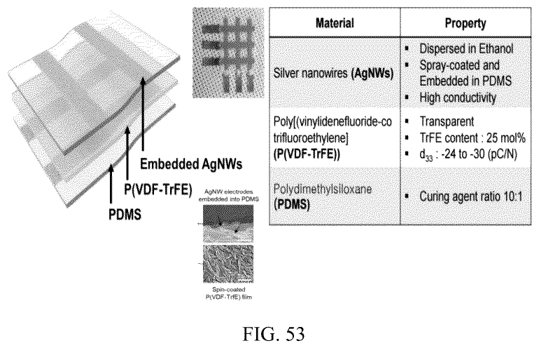

In one or some embodiments of the present application, a structure of different layers of a nano particle sensing patch is shown in FIG. 53.

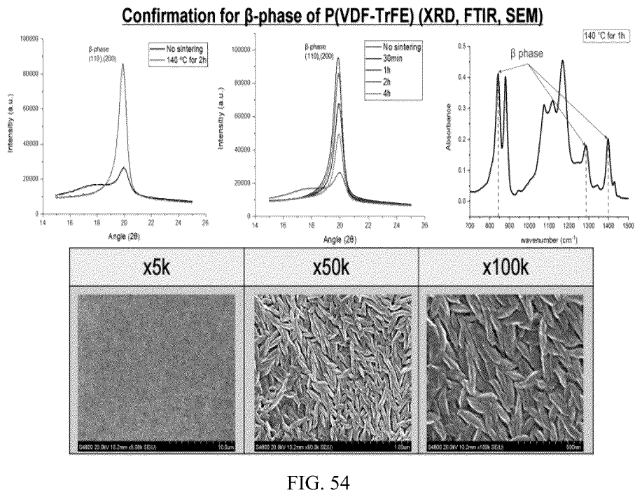

Further, FIG. 54 shows images of a nano particle sensing patch under different magnifications.

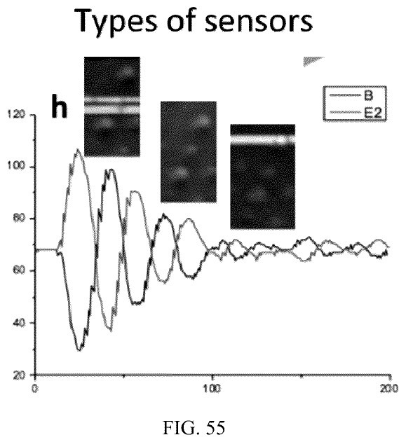

In one or some embodiments of the present application, the response characteristics of different types of sensors are shown in FIG. 55.

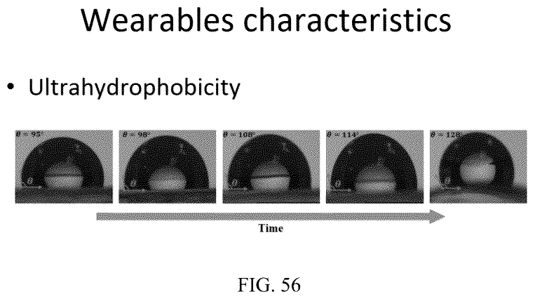

In one or some embodiments of the present application, characteristics of different wearable devices are shown in FIG. 56.

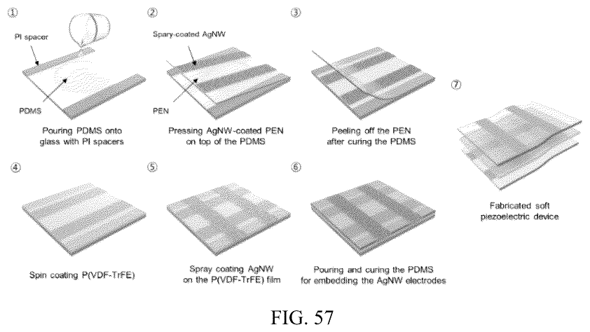

In an embodiment of the present application, as shown in FIG. 57, a method for preparing a flexible magnetoelectric piezoelectric switch apparatus, including: the flexible magnetoelectric piezoelectric switch apparatus is obtained by coupling a transducer, a thin film flexible substrate, and a sensor sequentially.

In summary, the flexible magnetoelectric piezoelectric switch apparatus provided by the present application achieves multi-field coupling by optimizing nano particle deposition and MENPs of the core-shell structure, improves sensitivity and energy efficiency, and reduces power consumption. The apparatus has good mechanical flexibility, ultrahydrophobicity and a “driving-sensing-feedback” closed-loop system, which is suitable for wearable devices, medical sensors, etc. It has the potential for commercialization and can realize needle-free drug delivery and real-time physiological monitoring.

The above describes the preferred embodiments of the present disclosure and is not intended to limit the present disclosure. Any modification, equivalent replacement, and improvement made within the spirit and scope of the present disclosure shall fall within the protection scope of the present disclosure.

What is claimed is:

1. A flexible magnetoelectric piezoelectric switch apparatus, comprising a thin film flexible substrate, wherein the thin film flexible substrate comprises: a flexible substrate, a piezoelectric substrate, and magnetoelectric nano particles;
the magnetoelectric nano particles are of a core-shell structure, comprising a cobalt ferrite magnetic core and a barium titanate piezoelectric shell layer; and the magnetoelectric nano particles are dispersed on a surface of the piezoelectric substrate.

2. The flexible magnetoelectric piezoelectric switch apparatus according to claim 1, wherein preparation of the magnetoelectric nano particle comprises the following steps:
preparing the CoFe₂O₄ magnetic core by a hydrothermal method; and then coating a surface of the CoFe₂O₄ magnetic core with the BaTiO₃ shell layer to form the core-shell structure, and obtaining the magnetoelectric nano particle by cooling.

3. The flexible magnetoelectric piezoelectric switch apparatus according to claim 2, wherein a cooling rate is 14-52° C./min, and an average particle size distribution range of the magnetoelectric nano particles is 19-31 nm.

4. The flexible magnetoelectric piezoelectric switch apparatus according to claim 3, wherein CoFe₂O₄ particles are prepared by dissolving an aqueous solution of a soluble cobalt salt, a soluble iron salt, and polyvinylpyrrolidone in an aqueous solution of sodium borohydride, and performing heating and mixing;
a BaTiO₃ precursor solution is prepared by mixing an aqueous solution containing barium carbonate and a citric acid with an ethanol solution containing titanium isopropoxide and a citric acid; and
the magnetoelectric nano particles are obtained by dispersing the CoFe₂O₄ particles in the BaTiO₃ precursor solution, performing ultrasonic processing, drying, and calcination, and then controlling the cooling rate.

5. The flexible magnetoelectric piezoelectric switch apparatus according to claim 1, wherein the flexible substrate comprises a polydimethylsiloxane thin film, a silver nanowire network, and a polyvinylidene fluoride film;
the silver nanowire network is embedded in a surface of the polydimethylsiloxane thin film to form a silver nanowire network embedded polydimethylsiloxane thin film; and
a surface of the silver nanowire network embedded polydimethylsiloxane thin film is coated with the polyvinylidene fluoride film to form the flexible substrate.

6. The flexible magnetoelectric piezoelectric switch apparatus according to claim 1, wherein the piezoelectric substrate is a polyvinylidene fluoride-trifluoroethylene copolymer thin film.

7. The flexible magnetoelectric piezoelectric switch apparatus according to claim 1, wherein when the magnetoelectric nano particles are dispersed on the surface of the piezoelectric substrate, the magnetoelectric nano particles are demagnetized.

8. The flexible magnetoelectric piezoelectric switch apparatus according to claim 1, wherein the flexible magnetoelectric piezoelectric switch apparatus further comprises a transducer and a sensor.

9. A method for preparing a flexible magnetoelectric piezoelectric switch apparatus according to claim 8, wherein the flexible magnetoelectric piezoelectric switch apparatus is obtained by coupling a transducer, a thin film flexible substrate, and a sensor sequentially.

10. An application of a flexible magnetoelectric piezoelectric switch apparatus obtained by a method for preparing according to claim 9 in a wearable device, a medical sensor, and a drug delivery system.

11. A wearable patch, comprising a flexible magnetoelectric piezoelectric switch apparatus obtained by a method for preparing according to claim 9.
